(19) United States
(12) Patent Application Publication
(10) Pub. No.: US 2021/0267669 A1
(43) Pub. Date: Sep. 2, 2021

(54) DEVICE FOR TREATING GASTROESOPHAGEAL REFLUX DISEASE AND METHOD FOR TREATING GASTROESOPHAGEAL REFLUX DISEASE

(71) Applicant: OLYMPUS CORPORATION, Tokyo (JP)

(72) Inventors: Nobuko MATSUO, Tokyo (JP); Keiichi SATO, Tokyo (JP); Yoshisane NAKAMURA, Tokyo (JP)

(73) Assignee: OLYMPUS CORPORATION, Tokyo (JP)

(21) Appl. No.: 16/804,804

(22) Filed: Feb. 28, 2020

(51) Int. Cl. A61B 18/14

(52) U.S. Cl. CPC A61B 18/1482; A61B 2018/00982; A61B 18/1445

(57) ABSTRACT

In a method for treating gastroesophageal reflux disease, an insertion portion of an endoscope is inserted from a cardia into a stomach. A distal end portion can be bent to position a cardiac region and a cautery unit in a field of view of an observation mechanism. The cautery unit can be brought into contact with a first region of a stomach wall around the cardia to cauterize the first region without removing a mucosal layer thereof while the cautery unit and a proximal portion of the insertion portion proximal of the bend are separated from each other by a predetermined distance or more. A position of the cautery unit with respect to the stomach wall can be changed while the separation state is maintained to bring the cautery unit into contact with and cauterize a second region of the stomach wall without removing a mucosal layer thereof.

### BACKGROUND

As a treatment for gastroesophageal reflux disease (GERD), an oral treatment with a gastric acid secretion inhibitor and surgery such as laparoscopic Nissen surgery are known.

The oral treatment is not a fundamental treatment, requires long-term administration, and may not improve symptoms.

A radical cure can be expected by the surgery. However, in the surgery, invasiveness is high. Since GERD is not a malignant disease such as a tumor, it is desirable that the invasiveness associated with treatment is as small as possible.

Various endoscopic treatments are considered as options other than oral treatment and surgical treatment. As one of the endoscopic treatments, a procedure is known in which a mucous membrane near a gastroesophageal junction is excised such that a scar is generated at an excised site and stricture is generated.

### SUMMARY

The present disclosure relates to a device for treating gastroesophageal reflux disease and a method for treating gastroesophageal reflux disease.

According to an aspect of the present disclosure, there is provided a method for treating gastroesophageal reflux disease.

In this treatment method, an insertion portion of an endoscope, which has an observation mechanism and a bendable distal end portion, is inserted from a cardia into a stomach, and the bending portion is bent to position a cautery unit and a cardiac region in a field of view of the observation mechanism.

The cautery unit can be brought into contact with a first region of a stomach wall around the cardia to cauterize the first region without removing a mucosal layer of the first region while the cautery unit and a proximal portion of the insertion portion, which is proximal of the bend, are separated from each other by a predetermined distance or more.

A position of the cautery unit with respect to the stomach wall can be changed while the separation state between the cautery unit and the proximal portion is maintained to bring the cautery unit into contact with and cauterize a second region of the stomach wall without removing a mucosal layer of the second region.

According to another aspect of the present disclosure, there is provided a device for treating gastroesophageal reflux disease.

This treatment device includes a fixing unit that is mountable on an insertion portion of an endoscope, and can maintain a relative position between the insertion portion and a cautery unit for cauterizing living tissue.

The treatment device can be applied to the treatment method discussed above.

### BRIEF DESCRIPTION OF THE DRAWINGS

FIG. 1 is a view illustrating a state in which a gastroesophageal junction is observed with an endoscope inserted into a stomach in a basic procedure according to the present disclosure.

FIG. 2 is a view illustrating an example of a treatment region.

FIG. 3 is a view illustrating an example of a treatment region that is marked.

FIG. 4 is a view illustrating an example of a lifted treatment region.

FIG. 5 is a schematic cross-sectional view of a stomach wall.

FIG. 6 is a view illustrating a state in which a treatment tool protruding from an endoscope is in contact with a site of the treatment region close to a greater curve.

FIG. 7 is a view illustrating a state in which the treatment tool protruding from the endoscope is in contact with a site of the treatment region close to a lesser curve.

FIG. 8 is a view illustrating an example of a damaged region.

FIG. 9 is a view illustrating an example of an incomplete stricture formed according to a repair of the damaged region.

FIG. 10 is a cross-sectional view taken along line I-I of FIG. 9.

FIG. 11 is a view illustrating a treatment device according to an exemplary embodiment.

FIG. 12 is an enlarged view illustrating a connection site between a guide and a support member in the treatment device.

FIG. 13 is a partial cross-sectional view of a fixing unit according to the treatment device.

FIG. 14 is a view illustrating a step of a basic procedure using the treatment device.

FIG. 15 is a view illustrating a step when the treatment device is used.

FIG. 16 is a view illustrating an exemplary connection structure of a cautery unit.

FIG. 17 is a view illustrating an exemplary fixing unit.

FIG. 18 is a partially enlarged view of an exemplary guide.

FIG. 19 is a partial cross-sectional view of an exemplary fixing unit.

FIG. 20 is a view illustrating a mode of operation of the exemplary fixing unit.

FIG. 21 is a view illustrating an exemplary fixing unit.

FIG. 22 is a view illustrating an exemplary fixing unit mounted on an endoscope.

FIG. 23 is a view illustrating a step when an exemplary fixing unit is used.

FIG. 24 is a view illustrating an exemplary fixing unit attached to an endoscope.

FIG. 25 is a view illustrating a step when an exemplary fixing unit is used.

FIG. 26 is a view illustrating an exemplary treatment device mounted on an endoscope.

FIG. 27 is a view illustrating a step when an exemplary treatment device is used.

FIG. 28 is a view illustrating an exemplary treatment device.

FIG. 29 is a view illustrating a step when an exemplary treatment device is used.

FIG. 30 is a view illustrating a step when an exemplary treatment device is used.

### DETAILED DESCRIPTION

A flow of a procedure (hereinafter, may be simply referred to as a “basic procedure”) according to the present disclosure will be described.

First, a physician inserts an endoscope through a natural opening such as a mouth or nose of a subject (insertion step), and moves a distal end portion of the endoscope into a stomach (gastrointestinal tract). A cap may be attached to the distal end portion of the endoscope.

As the endoscope, a known flexible endoscope can be used. The known endoscope includes an elongated insertion portion and has a bending portion which can be operated to bend at a distal end side of the insertion portion. An observation mechanism such as a light receiving window and an image sensor is provided at the distal end portion of the insertion portion and can observe a subject. The insertion portion has a channel through which a treatment tool passes, and the treatment tool which has passed through the channel protrudes from the distal end of the insertion portion.

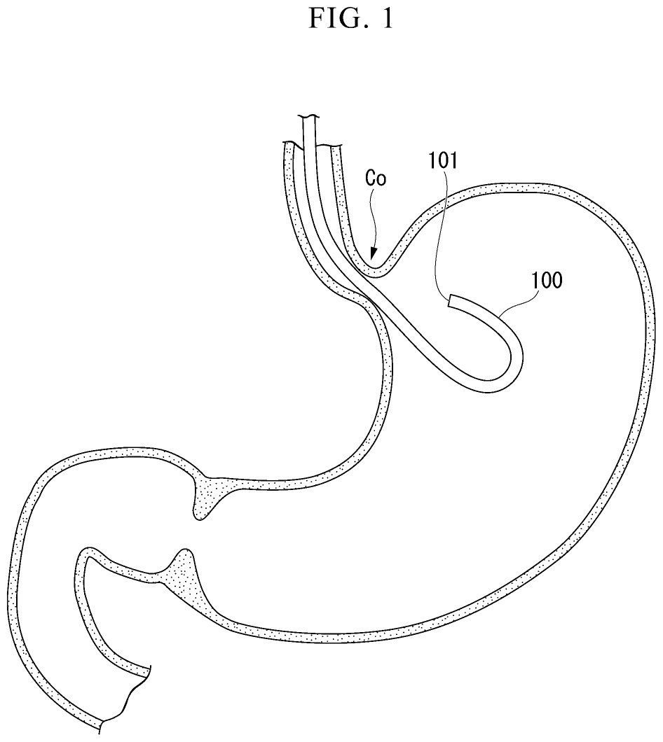

Next, the physician operates the bending portion of the endoscope to bend. As illustrated in FIG. 1, the physician points a distal end 101 of an endoscope 100 toward a cardia Co and captures a gastroesophageal junction around the cardia Co in a field of view of the endoscope 100. The physician determines a treatment region for performing a cautery treatment described later while observing the gastroesophageal junction (Step A).

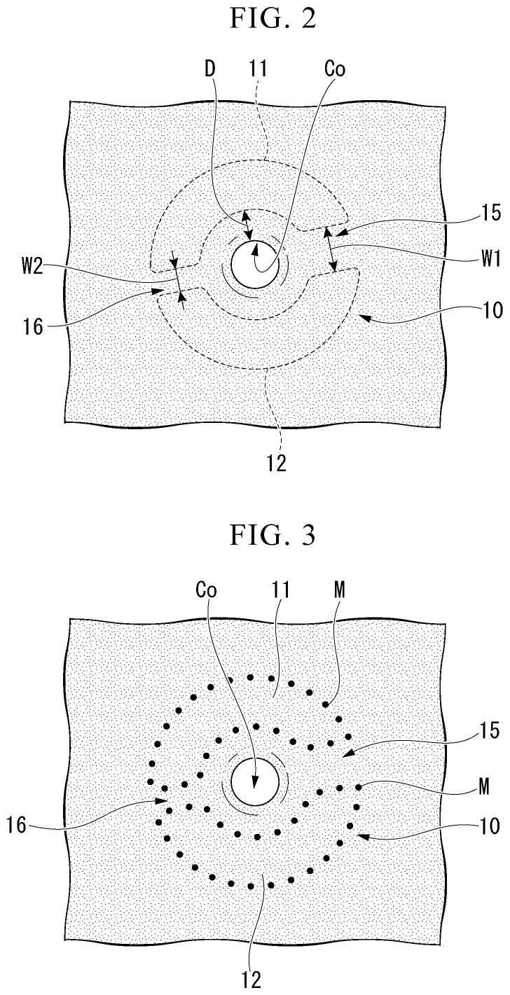

FIG. 2 illustrates an example of the treatment region. As illustrated in FIG. 2, the treatment region 10 has a shape in which C-shaped or U-shaped first region 11 and second region 12 face each other across the cardia Co. The first region 11 is located on an anterior wall side of the stomach. The second region 12 is located on a posterior wall side of the stomach. The first region 11 and the second region 12 extend in a circumferential direction of the gastroesophageal junction.

The two sub-regions of the first region 11 and the second region 12 are disposed to face each other. Accordingly, a first boundary portion (first undamaged region) 15 on a greater curve side and a second boundary portion (second undamaged regions) 16 on a lesser curve side are located respectively. The first boundary portion 15 extends along the greater curve. The second boundary portion 16 extends along the lesser curve.

A width (size in the circumferential direction of the gastroesophageal junction) W1 of the first boundary portion 15 is larger than a width W2 of the second boundary portion 16. For example, the width W1 is 10 to 20 millimeters and the width W2 is 5 to 10 millimeters. In the following description, the first region 11 and the second region 12 may be collectively referred to as “sub-regions 11 and 12”.

Each of the sub-regions 11 and 12 is apart from the cardia Co by a predetermined distance D and a gap exists between the cardia Co and the sub-regions 11 and 12. For example, the predetermined distance D is 5 to 10 millimeters. The predetermined distance D of the first region 11 and the predetermined distance D of the second region 12 may not to be the same.

For example, each of widths of the sub-regions 11 and 12 extending in an arc along the circumferential direction of the gastroesophageal junction is 10 to 20 millimeters. The width may be constant or may vary from site to site. Further, the width of the first region 11 and the width of the second region 12 may be different from each other.

Next, the physician protrudes the treatment tool from the endoscope 100 and marking M around the treatment region 10 determined as illustrated in FIG. 3 using the treatment tool (Step B). The marking M is performed by locally cauterizing a mucous membrane at peripheral portions of the sub-regions 11 and 12. As the treatment tool for marking M, for example, a high-frequency knife or a heat probe can be used.

The marking M does not need to be over the entire periphery of the treatment region, and may be at a plurality of spaced apart locations. In a treatment method of the present embodiment, since the first boundary portion 15 and the second boundary portion 16 play an important role, the marking M may be provided only around the first boundary portion 15 and the second boundary portion 16.

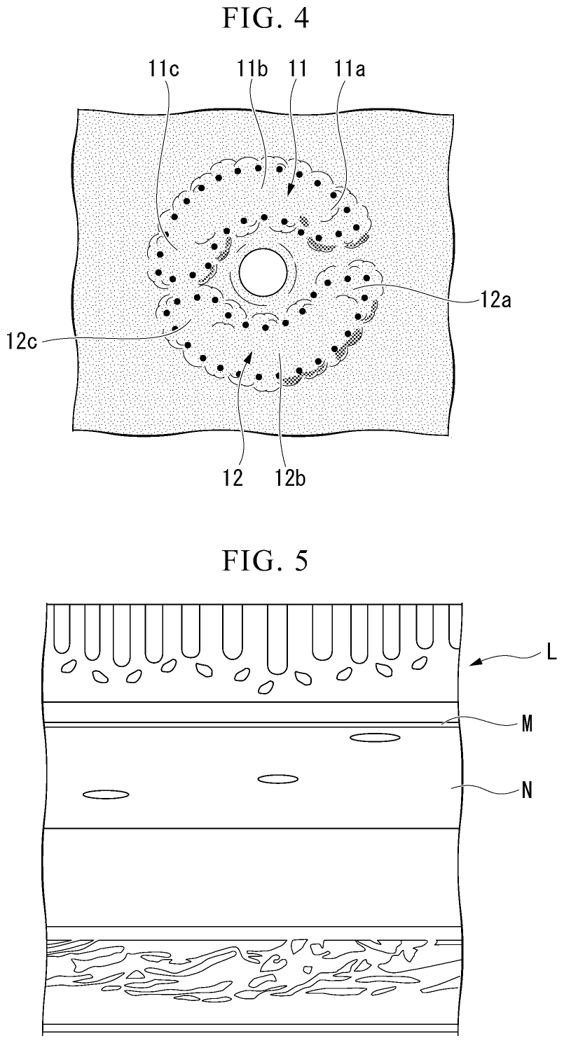

Next, the physician injects a liquid into a submucosal layer of each of the sub-regions 11 and 12 and lifts each of the sub-regions 11 and 12 as illustrated in FIG. 4 (Step C). As the liquid to be injected, physiological saline or the like can be used. It is preferable to color the liquid with a dye used in a gastrointestinal endoscope such as an indigo blue, because a degree of cauterization can be easily ascertained in a later step. The liquid can be injected using a local injection needle or the like for an endoscope.

Next, the physician cauterizes sites 11a and 12a (refer to FIG. 4) of a gastric mucosa in the sub-regions 11 and 12 close to a greater curve using the treatment tool protruding from the endoscope. In this case, unlike a known procedure, only the cauterization is performed without removing the mucous membrane. The degree of the cauterization is set such that a mucosal basal layer or a submucosal layer is damaged. FIG. 5 illustrates a schematic cross-sectional view of a stomach wall. A mucosal basal layer M is a layer which is a portion of a mucosal layer L and includes a boundary surface which is in contact with a submucosal layer N. The mucosal basal layer M may be referred to as basement membrane.

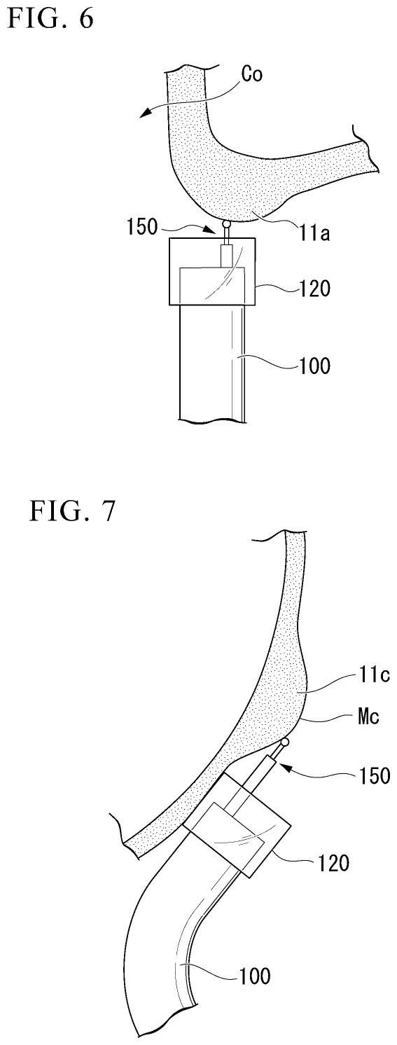

The lifted sites 11a and 12a have a positional relationship that easily faces the endoscope due to an anatomical shape of a gastric fundus. Accordingly, by moving the treatment tool 150 while fixing the endoscope 100, as illustrated in FIG. 6, the treatment tool 150 can come into contact with the mucous membrane of the target sites 11a and 12a.

The physician twists or bends the endoscope 100 to repeat the cauterization while changing the position of the distal end of the treatment tool 150, and thus, cauterizes the entire mucous membrane in the sites 11a and 12a. Since the sites 11a and 12a are close to the gastric fundus, in this Step, a region of the treatment region 10 on the gastric fundus side is cauterized (step D).

Next, the physician uses the treatment tool protruding from the endoscope to cauterize at the sites 11b and 12b close to the front and posterior walls and the sites 11c and 12c (refer to FIG. 4) close to the lesser curve in the gastric mucosa in the sub-regions 11 and 12 (Step E). In this case, it should be noted that only the cauterization is performed without removing the mucous membrane. The treatment tool to be used may be similar to the cauterization of the sites 11a and 12a.

Due to the anatomical shape of the stomach, the sites 11b, 12b, 11c, and 12c are difficult to face the endoscope 100 even if the sites are lifted. As a result, the treatment tool 150 protruding from the endoscope 100 approaches the mucous membrane in a state of being parallel or nearly parallel to the stomach wall.

Therefore, in a case where a high-frequency knife is used as the treatment tool 150, the treatment tool 150 may move forward along the mucous membrane Mc in a state where the endoscope 100 is fixed along the stomach wall as illustrated in FIG. 7, and the cautery may be performed while the treatment tool 150 moves rearward. When the treatment is performed in this procedure, the mucous membrane can be appropriately cauterized using discharge from the distal end of the treatment tool 150. This is an example of a cauterization procedure, and the cauterization may be performed in another procedure. In FIGS. 6 and 7, a cap 120 is attached to the distal end of the endoscope 100. It is not essential to mount a cap in the treatment method of the present embodiment. However, when the cap is mounted, it is possible to push the stomach wall while maintaining a good view of the endoscope. As a result, positioning of the treatment tool is facilitated.

When all the mucosal layers in the treatment region 10 are cauterized, the treatment region 10 becomes a cauterized damaged region while the mucosal layer remains. The physician removes the endoscope and terminates the procedure. After the cauterization, the entire treatment region may be observed from a bird's-eye view with an endoscope, and additional cauterization may be performed according to the observation result.

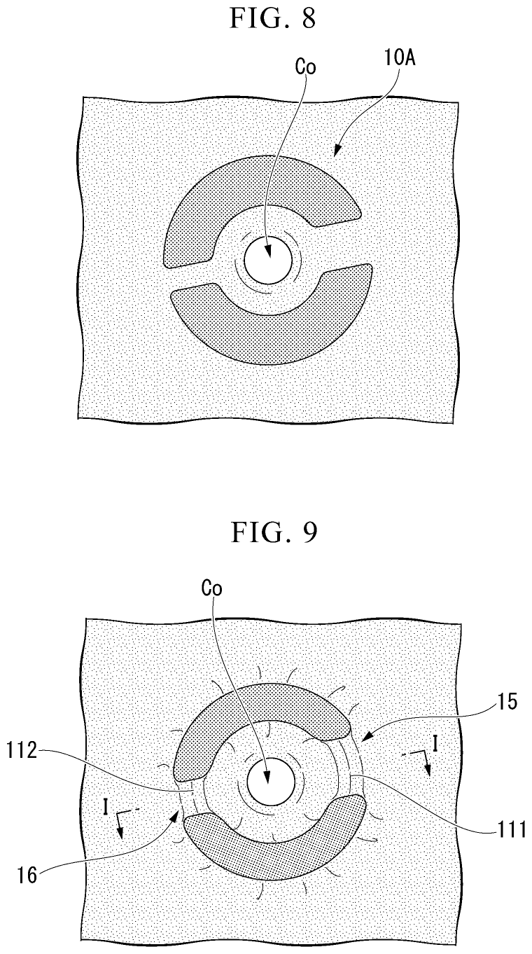

FIG. 8 illustrates an example of a damaged region 10A after the cauterization. The mucosal layer in the damaged region 10A remains because the mucosal layer is not removed. The gastric mucosa in the damaged region 10A is cauterized and damaged to reach the mucosal basal layer or the submucosal layer, and thereafter, is regenerated through a scar. When the mucous membrane is generated, a cautery site is shrunk due to scarring of the cautery site, and thus, the gastric mucosa around the treatment region is drawn toward the treatment region. As a result, the gastric mucosa is bent at the first boundary portion 15 and the second boundary portion 16, and as illustrated in FIG. 9, holds 111 and 112 are formed, which protrude while extending in the circumferential direction of the gastroesophageal junction.

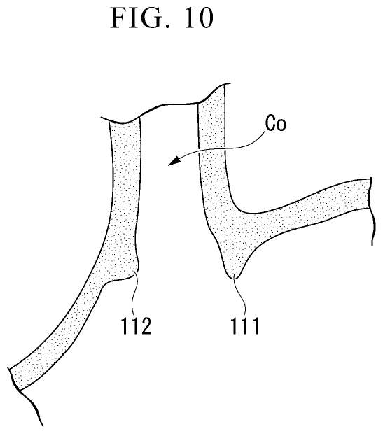

FIG. 10 is a schematic cross-sectional view taken along line I-I of FIG. 9. An His angle on the greater curve side is formed by the fold 111. The fold 112 functions as a valve for preventing reflux from the lesser curve side. In a subject to which the treatment method of the present disclosure is applied, incomplete stricture is formed at a cardiac region by the drawn gastric mucosa. As a result, the reflux of gastric contents is reduced, and symptoms of GERD are improved.

The above is the flow of the basic procedure. Since the basic procedure only involves cauterizing the treatment region 10 using the endoscope 100 and the treatment tool 150 inserted from a natural opening, the GERD treatment can be performed more easily than known procedures, such as those where the mucosal layer is excised. However, the basic procedure requires a cooperative operation between the endoscope 100 and the treatment tool 150. In addition, since an area which can be cauterized at one time by the above-described high-frequency knife is a small, steps D and E are likely to be complicated in conjunction with the above-described cooperative operation.

Therefore, there is room for the above-described basic procedure to be further simplified, and widespread use can be expected.

The treatment device and treatment method of the present disclosure contribute to further simplify the basic procedure. Hereinafter, each embodiment of the present disclosure will be described.

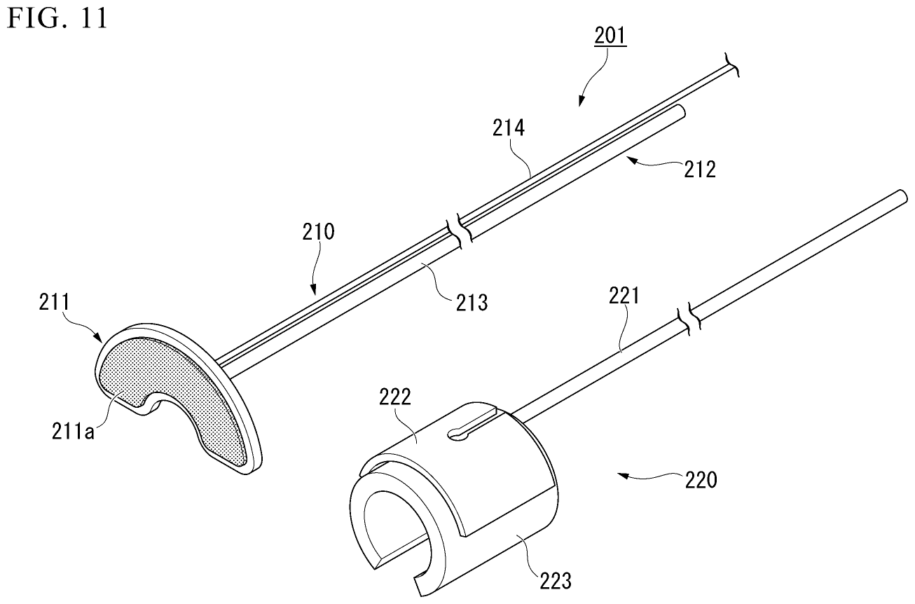

An exemplary embodiment of the present disclosure will be described with reference to FIGS. 11 to 23. FIG. 11 is a view illustrating a configuration of a treatment device 201 according to the present embodiment.

The treatment device 201 includes a cautery unit 210 and a fixing unit 220.

The cautery unit 210 includes a flexible heater 211 and an operation unit 212 which is connected to the heater 211.

The heater 211 has a cautery region 211a in the shape of the above-described first region 11 or second region 12 outside a channel, and is folded or rounded to pass through a channel of the endoscope 100 or an esophagus. As a basic configuration of the cautery region 211a, for example, an ablation electrode pattern described in Japanese Unexamined Patent Application, First Publication No. 2014-208303 can be adopted. By providing the ablation electrode pattern in the shape of the first region 11 or the second region 12 on an outer surface of a balloon, the balloon is deflated and passed through the channel, and the balloon is inflated outside the channel to constitute the heater 211 which can be returned to the shape of the first region 11 or the second region 12.

The above is an example of a specific configuration of the heater 211, and other configurations may be used.

The operation unit 212 includes an elongated operation member 213 and a wire 214. The operation member 213 is thick enough to pass through the channel, and has both flexibility which can follow a shape change in the body of the endoscope insertion portion and rigidity which can maintain its own shape outside the channel. As the operation member 213, for example, a wire formed of metal or resin can be used.

The wire 214 extends along the operation member 213. A front end of the wire 214 is connected to the heater 211 and supplies electric power for the cauterization to the heater 211. A rear end of the wire 214 is connected to a power source (not illustrated) outside the endoscope 100.

The fixing unit 220 has an elongated support member 221, a guide 222 which is attached to the support member 221, and a ring 223.

The support member 221 of the present embodiment is formed of a shape memory alloy such as a nickel titanium alloy and is a belt-shaped member having the same flexibility and rigidity as the operation member 213.

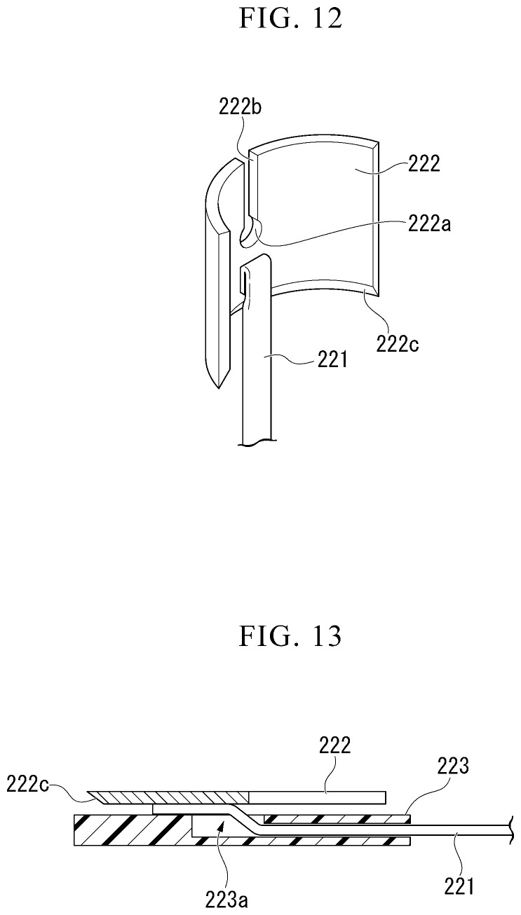

The guide 222 has a shape constituting a portion of a cylinder and includes a through hole 222a and a slit 222b extending from the through hole 222a to a periphery. As illustrated in FIG. 12, a distal end portion of the support member 221 has a curl of about 180 degrees, and an outer peripheral surface of the distal end portion and the guide 222 are joined to each other.

The ring 223 is a C-shaped member, and is mounted on the insertion portion of the endoscope to be held at a desired position of the insertion portion. As illustrated in cross section in FIG. 13, the ring 223 has an axially extending space 223a in a wall of the ring 223. The rear portion of the space 223a is open to a proximal end surface of the ring 223, and a front portion of the space 223a is bent and opens to an outer peripheral surface of the ring 223.

The support member 221 enters the space 223a from a proximal end side of the ring 223.

A flow of performing the basic procedure using the treatment device 201 configured as described above will be described.

First, the fixing unit 220 is mounted on the insertion portion of the endoscope 100. In this case, the ring 223 is mounted on a proximal side from the bending mechanism which can perform a bending operation. If necessary, the support member 221 is held at several places along the insertion portion using a C-shaped auxiliary member or the like. When the support member 221 is pulled, the curl of the distal end portion is extended, and the guide 222 is disposed along the outer peripheral surface of the ring 223 as illustrated in FIG. 13.

The insertion step and Steps A to C are performed in the same manner as described above using the endoscope 100 on which the fixing unit 220 is mounted.

Details of Steps D and E using the treatment device 201 will be described.

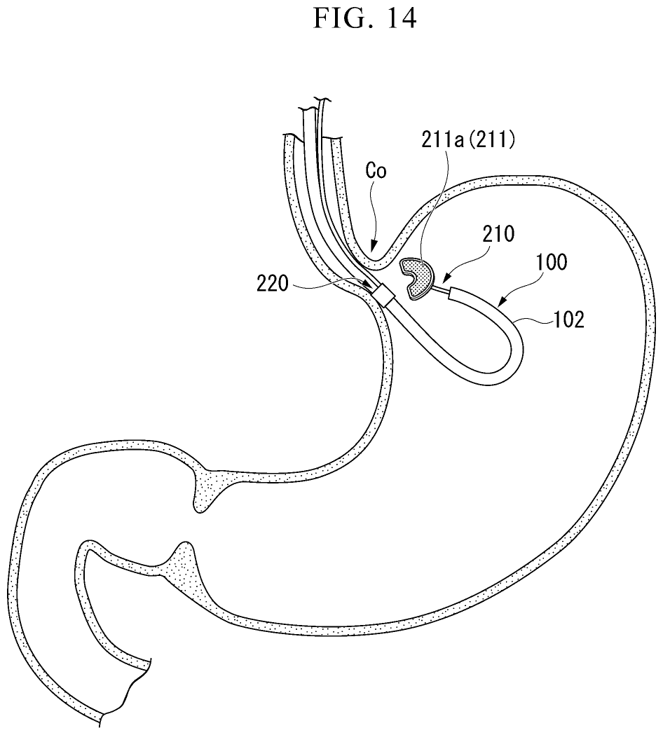

The physician removes the local injection needle and the like used in Step C from the endoscope 100, and causes the cautery unit 210 to pass through the channel. Thereafter, as illustrated in FIG. 14, the insertion portion 102 is curved to capture the cardia Co in a visual field, and the heater 211 of the cautery unit 210 protrudes from the endoscope 100 (Step d1). The cautery region 211a of the heater 211 which has exited the endoscope 100 is developed by a predetermined operation to have the shape of the first region 11 or the second region 12 (Step d2).

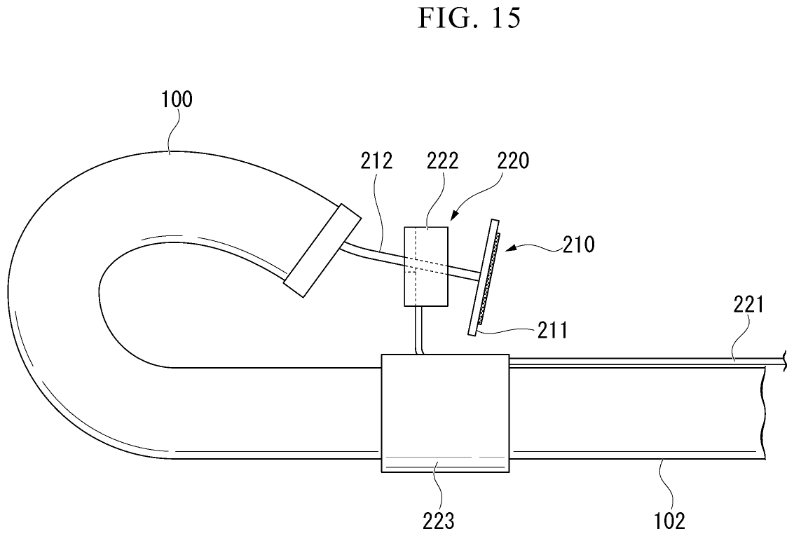

Next, the physician pushes the support member 221 of the fixing unit 220 forward. Then, the support member 221 protrudes outside the ring 223. In this case, due to the curl of the distal end portion of the support member 221, the guide 222 stands up substantially perpendicularly to the outer peripheral surface of the ring 223 as illustrated in FIG. 15. A distance between the guide 222 and the insertion portion 102 can be adjusted by changing a pushing amount of the support member 221.

The physician places the operation member 213 of the cautery unit 210 in the through hole 222a through the slit 222b of the guide 222 by a bending operation of the endoscope 100 or the like. Thereby, the operation member 213 is fixed to the guide 222, and a position of the heater 211 with respect to the endoscope 100, more specifically, a positional relationship with respect to the insertion portion 102 on the proximal end side of the bending portion is fixed and stable. Accordingly, the heater is located within the field of view of the endo scope 100. (Step d3).

A method for stably disposing the operation member 213 in the through hole 222a is not particularly limited. For example, a method of making a width of the slit 222b smaller than a diameter of the through hole 222a, or a method of meandering or bending the slit 222b can be exemplified.

When Step d3 ends, the cautery unit 210 (specifically, the heater 211) is held in a separation state separated from the insertion portion 102 on the proximal end side of the bending portion by a predetermined distance D.

Subsequently, the physician moves the endoscope 100 rearward or moves the operation member 213 forward so that the heater 211 comes into contact with the stomach wall around the cardia Co, and cauterizes the stomach walls of the first region 11 and the second region 12 by the heater 211 (Step d4). Since the cautery region 211a of the heater 211 has the shape of the first region or the second region, the first region and the second region can be easily formed only by heating the heater 211 while pressing the heater 211 against the stomach wall using the treatment device 201.

In the basic procedure, since the endoscope 100 is fastened to the cardia Co, a positional relationship between the insertion portion 102 on the proximal side from the bending mechanism and the cardia Co is substantially fixed. Therefore, a distance relationship between the heater 211 of which the relative position with respect to the endoscope 100 is stabilized by the fixing unit 220 and the cardia Co also is stabilized, and thus, Step d4 is easily performed.

In Step d4, when the heater 211 approaches or comes into contact with the first region 11 or the second region 12, if necessary, the insertion portion 102 may be rotated around a longitudinal axis of the insertion portion 102 or the operation member 213 may be rotated so that a position of the cautery unit 210 with respect to the stomach wall is adjusted (step d5). Further, the positional relationship may be changed or the cautery may be performed multiple times depending on a size of the first region or the second region, a cautery depth, or the like.

After Step d4 ends, the physician straightens the insertion portion 102 to remove the cautery unit 210 from the guide 222, and pulls the support member 221 so that the guide 222 follows the ring 223. As illustrated in FIG. 12, the guide 222 has an inclined surface 222c at a lower end of a surface to which the support member 221 is connected. When the support member 221 is pulled, the guide 222 approaches the ring 223, and the lower end of the guide 222 comes into contact with the ring 223. Then, the guide 222 falls down to the support member 221 side by the inclined surface 222c, and smoothly returns to a state illustrated in FIG. 13.

The endoscope 100 and the treatment device 201 are removed from the body of the subject and a series of procedures ends.

As described above, according to the treatment device 201 of the present embodiment, the position of the cautery unit 210 with respect to the endoscope 100 is fixed by the fixing unit 220, and a distance between an outer peripheral surface of the insertion portion 102 and the heater 211 is maintained to be constant. Therefore, when the endoscope 100 is operated after the endoscope 100 is fixed, the cautery unit 210 follows while maintaining the positional relationship between the endoscope 100 and the cautery unit 210.

As a result, the endoscope and the cautery unit can be easily cooperatively operated to cauterize a desired position of the stomach wall around the cardia, and the basic procedure can be performed more easily.

In the basic procedure, the rotation and the forward or rearward operation of the cautery unit 210 are also performed as necessary. However, the operations can be easily performed because the distance between the cautery unit 210 and the endoscope 100 is maintained within a predetermined range by the fixing unit 220.

In the present embodiment, various modifications are possible. Some of the modifications are exemplified below, but not all. In addition, the modifications can be appropriately combined.

The cautery unit is not limited to the cautery region having the shape similar to the first region or the second region. For example, the high-frequency knife used in Step B may be used as it is as the cautery unit. Also, in this case, the high-frequency knife is held by the fixing unit. Accordingly, Step D can be performed more easily than when the treatment device is not used.

For example, in a case where the cautery unit has a cautery region having a shape similar to a portion of the first region or the second region, after a range close to the site 11a of the same region is cauterized, the cautery region can be easily moved to other site (for example, a range close to the site 12a) of the same region simply by rotating the insertion portion 102. This operation is also included in the above-described Step d5.

The guide may not have a slit communicating with the hole. For example, a hole having a size enough to allow the cautery unit obtained by folding the heater to pass through may be formed in the guide and the cautery unit may pass through this hole to fix the position of the cautery unit.

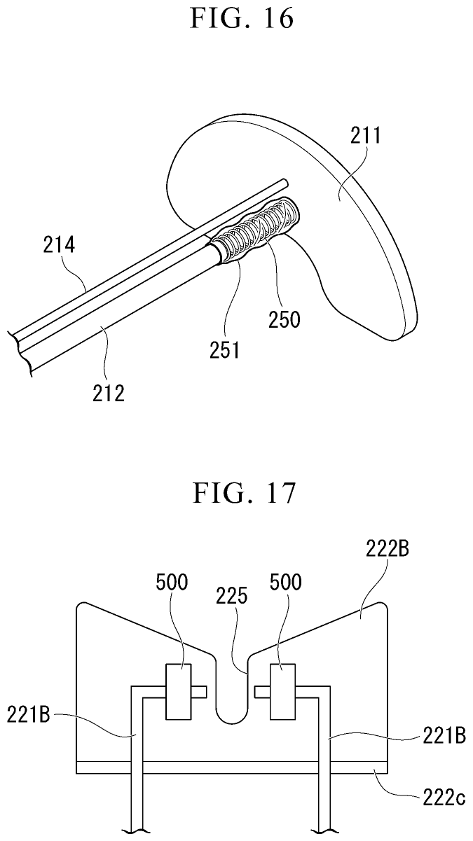

In the cautery unit, the heater and the operation member may be connected to each other by a ball joint, a coil spring, or the like so that a direction and posture of the heater with respect to the operation member can be adjusted. In the case where the heater and the operation member are connected to each other by a coil spring, as illustrated in FIG. 16, a tube 251 is disposed around the coil spring 250, and adjusted direction and posture can be temporarily fixed by sucking gas in the tube 251 and reducing a pressure.

The connection aspect between the guide and the support member in the fixing unit is not limited to the above-described example.

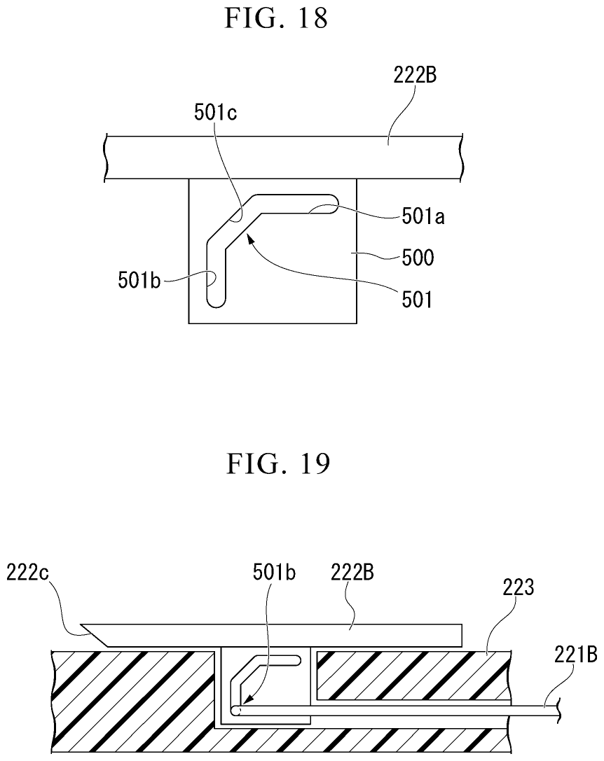

In a modification example illustrated in FIG. 17, a pair of support members 221B passes through connection portions 500 provided in a guide 222B. As illustrated in FIG. 18, a through groove 501 through which the support member 221B passes is formed in each of the connection portions 500. The through groove 501 includes a first region 501a substantially parallel to a plane direction of the guide 222B, a second region 501b substantially perpendicular to the plane direction of the guide 222B, and a third region 501c connecting the first region 501a and the second region 501b to each other.

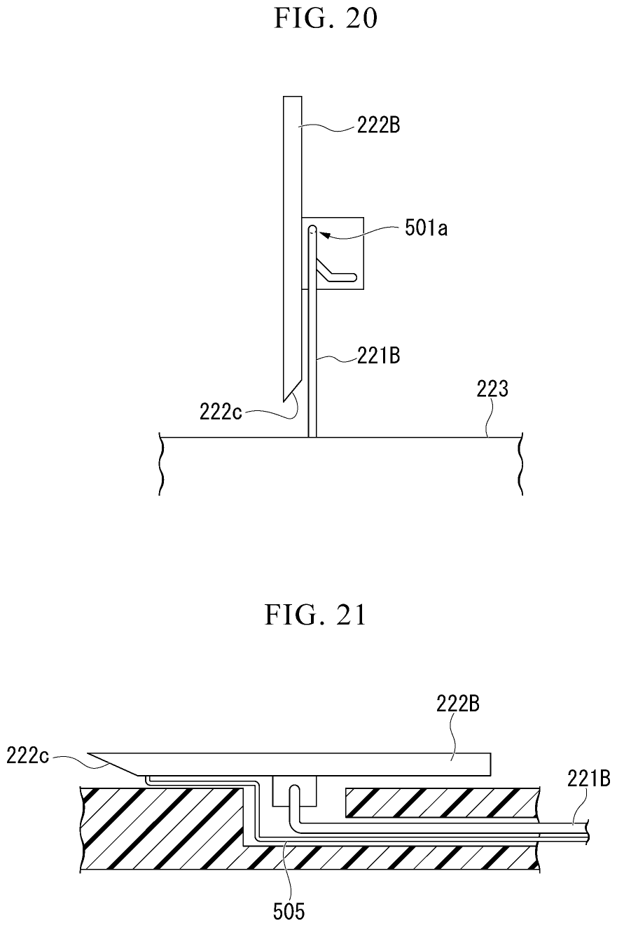

Since the support member 221B passing through the connection portion 500 can move freely in the through groove 501, when the support member 221B is pulled, the support member 221B moves to the second region 501b, and as illustrated in FIG. 19, the guide 222B can be held along the ring 223. When the support member 221B is moved forward, the support member 221B moves from the second region 501b to the first region 501a due to the gravity acting on the guide 222B. As a result, the guide 222B stands up with respect to the ring 223 as illustrated in FIG. 20.

Since the guide 222B has the inclined surface 222c similarly to the guide 222, the guide 222B rotates smoothly during storage and follows the insertion portion 102. In addition, as illustrated in FIG. 17, a slit 225 formed in the guide 222B is widened in the upper portion of the guide 222B. Accordingly, the cautery unit 210 can be easily introduced into the slit 225.

As illustrated in FIG. 21, a thread 505 may be connected to a lower end of the guide 222B. In this case, in a case where a posture of the guide 222B is not changed well due to a posture of the endoscope in the body, the posture of the guide 222B can be reliably changed by pulling the thread 505.

An aspect of the fixing unit is not limited to the above-described example.

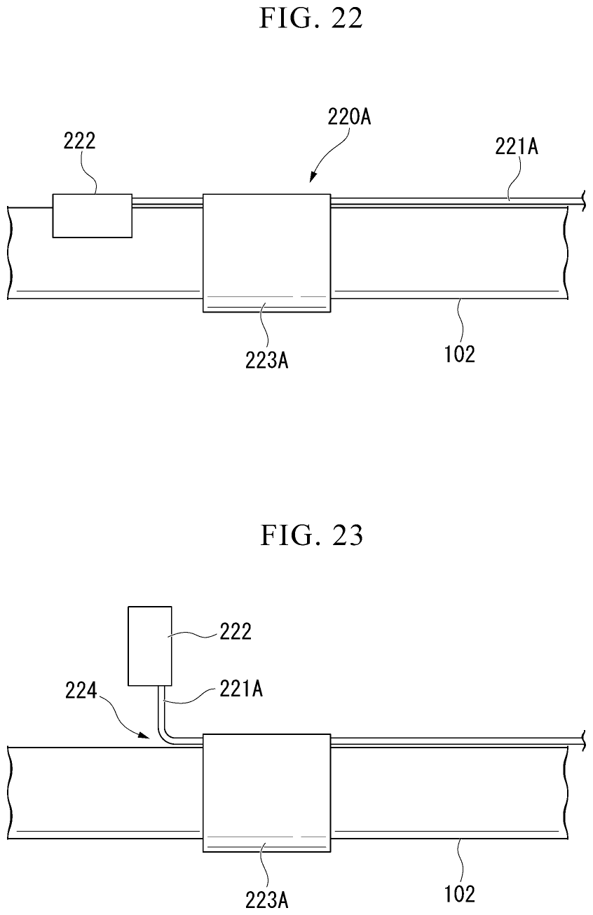

In a fixing unit 220A of a modification example illustrated in FIGS. 22 and 23, a support member 221A does not pass through a ring 223A, but passes between the ring 223A and the insertion portion 102. The support member 221A has a bent portion 224 to which a curl is attached at a right angle. In a state where the bent portion 224 is between the ring 223A and the insertion portion 102, as illustrated in FIG. 22, the guide 222 follows the insertion portion 102. However, when the support member 221A moves forward so as to move the bent portion 224 out of the ring 223A, the guide 222 stands up as illustrated in FIG. 23.

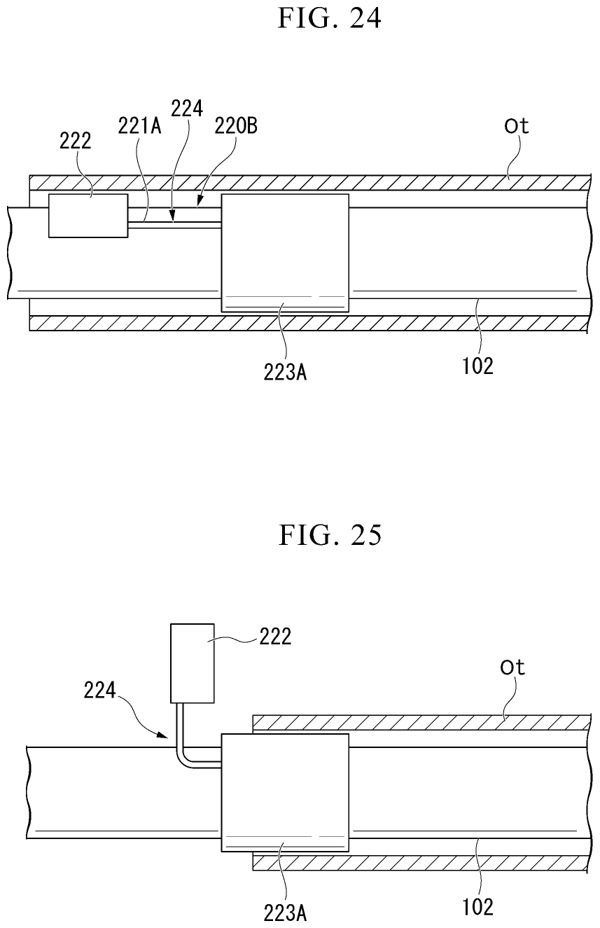

In a fixing unit 220B of the modification example illustrated in FIGS. 24 and 25, the support member 221A connects the ring 223A and the guide 222 to each other and does not extend to the proximal side. In this modification example, when the endoscope on which the fixing unit 220B is mounted is passed through the overtube Ot, the bent portion 224 is linearly deformed, and the guide 222 follows the insertion portion 102. When the overtube Ot moves rearward with respect to the endoscope, as illustrated in FIG. 25, a deformation of the bent portion 224 is released and the guide 222 stands up.

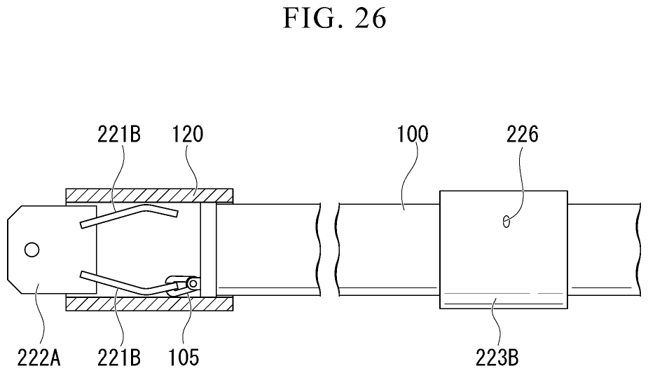

In a fixing unit, a support member and a ring may be disposed apart from each other and may be assembled in a body. In a modification example illustrated in FIG. 26, a cap 120 is mounted on the distal end portion of the endoscope 100, and a portion of the support member 221B and a portion of the guide 222A are disposed in the cap 120 apart from the ring 223B.

The support member 221B and the guide 222A disposed in the cap 120 are introduced into the body while being held by forceps 105. The ring 223B includes a hole 226 into which the pair of support members 221B can be inserted and a groove (not illustrated) in which the support members 221B can be disposed.

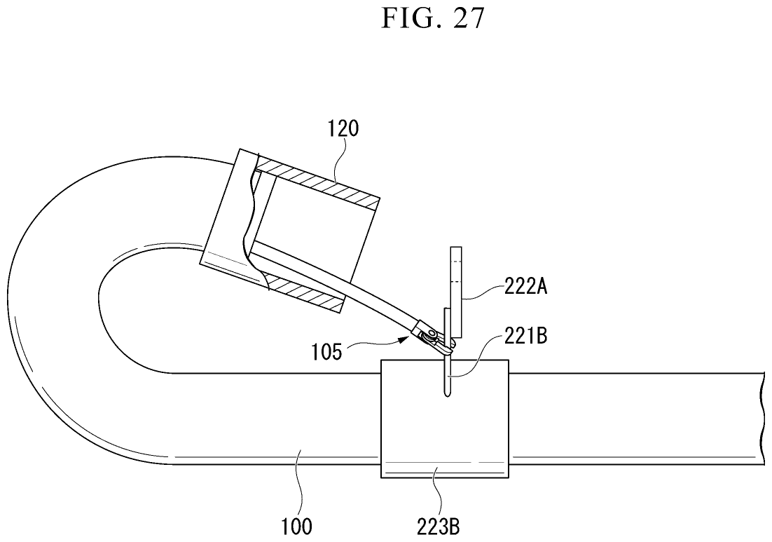

By attaching the support member 221B to the ring 223B using the forceps 105 as illustrated in FIG. 27 while observing the ring 223B by the curved endoscope 100, the fixing unit is assembled. After the basic procedure ends, the guide 222A and the support member 221B are detached from the ring 223B using the forceps 105 to disassemble the fixing unit, and are pulled out of the body together with the endoscope 100.

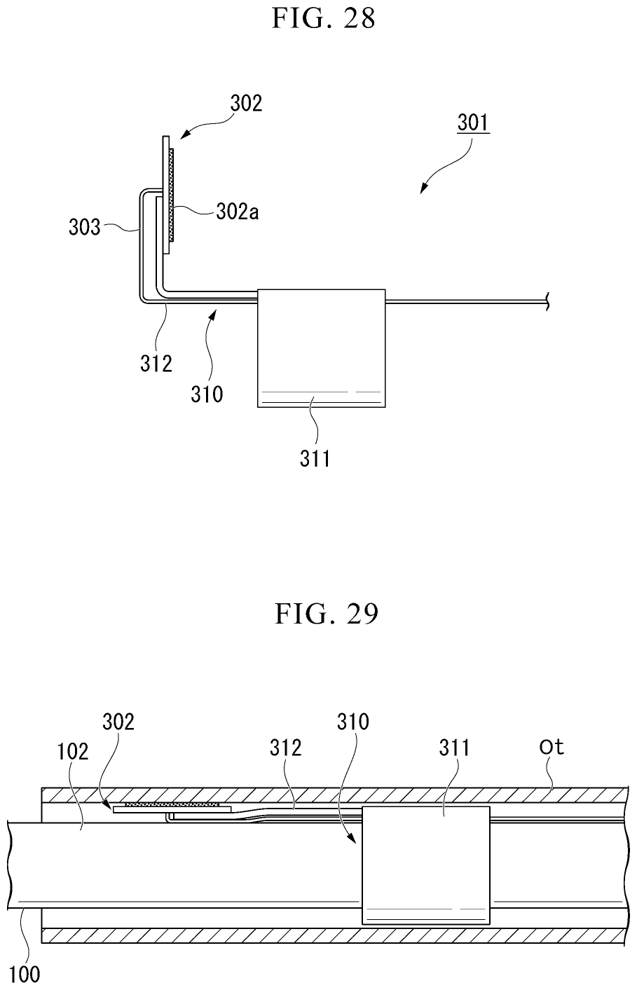

Another exemplary embodiment of the present disclosure will be described with reference to FIGS. 28 to 30. In the following description, the same components as those already described are denoted by the same reference numerals, and redundant description will be omitted.

FIG. 28 is a view illustrating a treatment device 301 of the present embodiment. The treatment device 301 includes a heater 302 as a cautery unit and a fixing unit 310. In the treatment device 301, the heater 302 and the fixing unit 310 are connected to each other.

The heater 302 has flexibility enough to follow an outer peripheral surface of an insertion portion of an endoscope. As the heater 302, the heater 211 of the above exemplary embodiment or other known configurations can be adopted. A wire 303 for supplying electric power is connected to the heater 302.

The fixing unit 310 has a ring 311 and a support member 312. The ring 311 has the same outer shape as the ring 223 of the above embodiment, and can be mounted on the insertion portion 102 of the endoscope 100. The support member 312 is an elongated member which is formed of a shape memory alloy and has the same physical properties as the support member 221 of the above embodiment. A front end of the support member 312 is connected to the heater 302. A rear end of the support member 312 is connected to the ring 311. An intermediate portion of the support member 312 has a curl. In the present embodiment, an angle of the curl is about 90 degrees, but the angle can be appropriately changed.

A cautery region 302a of the heater 302 is disposed on a surface on the ring 311 side.

Details of Steps D and E using the treatment device 301 will be described.

After Step C, a physician removes the endoscope 100, attaches the ring 311 to the insertion portion 102, and connects the wire 303 to a power source. A mounting position of the ring 311 is located on the proximal side from the bending mechanism, as in the above embodiment, and is specifically determined in Steps D and E based on a position where the heater 302 is to be disposed.

Next, the endoscope 100 on which the treatment device 301 is mounted passes through the overtube, and the heater 302 is positioned inside the overtube Ot as illustrated in FIG. 29. The heater 302 is pressed against the inner surface of the overtube Ot and is held along the outer peripheral surface of the insertion portion 102, and in the support member 312, the curl is extended.

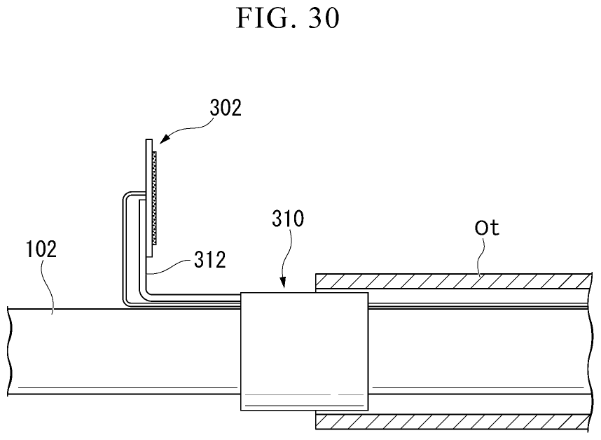

The physician inserts the overtube Ot and the endoscope 100 again into a target body. When the insertion portion 102 is curved and the cardia Co is captured in the field of view of the endo scope 100 by the same operation as in the above embodiment, the overtube Ot moves rearward to locate the heater 302 and the support member 312 out of the overtube Ot. Then, the support member 312 is bent again by the curl, and as illustrated in FIG. 30, the heater 302 stands up with respect to an outer peripheral surface of the insertion portion (Step d3). Since a positional relationship between the heater 302 and the insertion portion 102 is maintained by the fixing unit 310, the physician operates the endoscope 100 and can easily perform Step d4.

The treatment device 301 of the present embodiment can move the cautery unit to a desired position only by operating the endoscope to cauterize a desired position of the stomach wall around the cardia, and can more easily perform the basic procedure.

Since the cautery unit and the fixing unit are connected to each other in advance, it is not necessary to mount the cautery unit on the fixing unit. Moreover, the positional relationship between the cautery unit and the endoscope insertion portion can be maintained only by moving the overtube Ot rearward. Thereby, the operation is further simplified.

The following modifications may be made to the configuration of the present embodiment.

The mounting of the treatment device 301 and the insertion thereof into the overtube Ot may be performed before Step A.

A connection site between the heater 302 and the support member 312 may have a telescopic structure or the like so that a distance between the cautery unit and the insertion portion may be adjusted. For example, an adjustment operation can be performed using the forceps passing through the endoscope or the like.

The ring 311 may be attached to the overtube Ot, and the endoscope 100 and the overtube Ot may pass through the second overtube. In this case, by moving the second overtube rearward, a positional relationship between the cautery unit and the overtube Ot is maintained, and by operating the overtube Ot, Step d4 can be performed.

This modification example has the advantage that Step d4 can be performed by moving only the cautery unit without changing the field of view of the endoscope.

While preferred embodiments have been described and illustrated above, it should be understood that these are exemplary and are not to be considered as limiting. Additions, omissions, substitutions, and other modifications can be made without departing from the spirit or scope of the present disclosure. Accordingly, the disclosure is not to be considered as being limited by the foregoing description, and is only limited by the scope of the appended claims.

In the treatment method of the present embodiment, the position of the treatment region and the order in which the treatment region is cauterized can be appropriately changed. For example, the sites 11a, 11b, and 11c may be cauterized in any order. The first region 11 and the second region 12 may be cauterized in parallel, or one of the first region 11 and the second region 12 may be cauterized before the other.

Since the cauterization in the treatment method of the present embodiment does not reach a muscular layer, the treatment method of the present embodiment can be performed without lifting. That is, Step C may be omitted. In a case where the overtube is not used, the insertion portion of the endoscope may pass through a tubular balloon to inflate the balloon, thereby facilitating a twisting operation of the endoscope in Steps D, E, or the like.

1. A method for treating gastroesophageal reflux disease, the method comprising:
inserting an insertion portion of an endoscope from a cardia into a stomach, the insertion portion including a distal end portion that is bendable, and an observation mechanism having an image sensor;
bending the distal end portion to form a bend therein and position a cautery unit and a cardiac region in a field of view of the observation mechanism, the cautery unit being configured to cauterize living tissue;
operating the insertion portion or the cautery unit to bring the cautery unit into contact with a first region of a stomach wall around the cardia in a separation state in which the cautery unit and a proximal portion of the insertion portion that is proximal of the bend in the distal end portion are separated from each other by a predetermined distance or more;
cauterizing the first region while leaving a mucosal layer of the first region;
operating the insertion portion while maintaining the separation state to change a position of the cautery unit with respect to the stomach wall; and
operating the insertion portion or the cautery unit while maintaining the separation state to bring the cautery unit into contact with a second region of the stomach wall that is different from the first region and cauterizing the second region while leaving a mucosal layer of the second region.

2. The method according to claim 1, wherein:
the first region and the second region include a mucosal layer near the cardia of an inner wall of the stomach, and
the predetermined distance is equal to a distance of a gap extending between: (i) each of the first region and the second region, and (ii) the cardia.

3. The method according to claim 2,
wherein the first region and the second region each extend only partially in a circumferential direction of the stomach.

4. The method according to claim 3, wherein:
the first region is an arc-shaped region formed on an anterior wall of the stomach, and the second region is an arc-shaped region formed on a posterior wall side of the stomach,
a first undamaged region where the mucosal layer is not cauterized is located between the first region and the second region on a greater curvature side of the stomach,
a second undamaged region where the mucosal layer is not cauterized is located between the first region and the second region on a lesser curve side of the stomach, and
a size of the first undamaged region in the circumferential direction is larger than a size of the second undamaged region in the circumferential direction.

5. The method according to claim 1,
wherein the cautery unit is attached to an outer surface of the proximal portion of the insertion portion during the cauterization of the first region and the second region.

6. The method according to claim 1,
wherein the insertion portion is rotated around a longitudinal axis thereof to bring the cautery unit into contact with the second region.

7. A device for treating gastroesophageal reflux disease, the device comprising:
a fixing unit configured to be mounted on an insertion portion of an endoscope and maintain a relative position between the insertion portion and a cautery unit that is configured to cauterize living tissue.

8. The device according to claim 7, wherein:
the fixing unit includes:
a guide configured to be attached to and detached from the cautery unit, and
a support connected to the guide,
wherein the guide is configured to be moved with respect to an outer peripheral surface of the insertion portion by movement or deformation of the support.

9. The device according to claim 8, wherein the fixing unit is configured to change a distance between the guide and the outer peripheral surface of the insertion portion such that the cautery unit can be attached to the guide.

10. The device according to claim 8, wherein fixing unit further comprises a ring that is configured to be mounted on the outer peripheral surface of the insertion portion, and is coupled to the support.

11. The method according to claim 5, wherein the cautery unit protrudes from a distal end of the insertion portion after the insertion portion is inserted into the stomach.

12. The method according to claim 11, further comprising attaching the cautery unit protruding from the distal end of the insertion portion to the outer surface of the proximal portion after the insertion portion is inserted into the stomach to maintain the separation state.

13. The method according to claim 12, wherein the cautery unit is attached to the proximal portion by a fixing unit mounted on the proximal portion of the insertion portion.

14. The method according to claim 13, wherein the cautery unit is attached to the proximal portion by:
operating the insertion portion or a support comprised by the fixing unit to move the support such that at least a portion of the support extends in a direction away from the outer surface of the proximal portion, and
capturing a portion of the cautery unit within a guide coupled to a distal end of the support.

15. The method according to claim 1, wherein the insertion portion is inserted into the stomach with the cautery unit mounted to an outer surface of the insertion portion.

16. The method according to claim 1, wherein the cautery unit protrudes from an outer surface of the proximal portion of the insertion portion during the cauterization of the first region and the second region, and does not protrude from a distal end of the insertion portion.
